(19) United States
(12) Patent Application Publication
(10) Pub. No.: US 2009/0130179 A1
(43) Pub. Date: May 21, 2009

(54) CONFECTIONERY COMPOSITIONS INCLUDING MARKINGS FOR DELIVERY OF ACTIVES

(75) Inventors: Jamileh Lakkis, Barcelona (ES); April Bowser, Huntersville, NC (US); Thomas Byrczek, New York, NY (US); Matthew Allan Beam, Califon, NJ (US)

Correspondence Address: HOFFMANN & BARON, LLP, 6900 JERICHO TURNPIKE, SYOSSET, NY, 11791, US

(73) Assignee: CADBURY ADAMS USA LLC, Parsippany, NJ (US)

(21) Appl. No.: 12/272,085

(22) Filed: Nov. 17, 2008

Related U.S. Application Data
(60) Provisional application No. 60/988,522, filed on Nov. 16, 2007.

(51) Int. Cl. A61K 9/68; A23L 1/48; A61K 8/18; A23G 4/06; A61Q 11/00

(52) U.S. Cl. 424/440; 426/87; 424/49; 424/48; 426/3

(57) ABSTRACT

The present invention relates to a confectionery composition including a confectionery region and a marking on the exterior surface of the region, where the marking includes at least one active agent. A method of manufacturing the confectionery composition and of delivering an active agent using the confectionery composition is also provided.

### CROSS-REFERENCE TO RELATED APPLICATIONS

The present invention claims priority to U.S. Provisional Application No. 60/988,522, filed Nov. 16, 2007, the entire contents of which are incorporated herein by reference.

### FIELD

The present invention relates to confectionery compositions which provide an enhanced delivery of active agents in the mouth when consumed. In particular, the present invention provides confectionery compositions including a confectionery region and a marking on the exterior surface thereof. The marking includes an active agent, which can be readily released into the oral cavity upon consumption.

### BACKGROUND

Although it is known to add active agents to a confectionery composition directly, such active agents are not released very readily into the oral cavity upon consumption. Active agents have been added to various areas in a confectionery composition, such as, for example, in a coating, a center-fill region, or in other regions. However, addition of active agents to these regions incorporates the active agent throughout the entire region, which may lead to difficulties in adding specific amounts of active agent per confectionery composition, and further may create difficulties in releasing the active agent while the confectionery is chewed or otherwise ingested. Further, addition of the active agent in the region as a whole may undesirably result in confectionery pieces that contain too much active agent.

There is presently a need for a confectionery composition, which includes an active agent added in specific and limited areas of the confectionery composition, such as by being formed in markings on the confectionery composition's exterior surface. Confectionery compositions described herein allow for an efficient delivery of a small amount of active agents, and allow for a control of the delivery of active agents which may interact with each other. Further, the confectionery compositions described herein allow for a faster release of active agents than with existing compositions.

### SUMMARY

In some embodiments, there is provided a confectionery composition including a confectionery region and a marking on the exterior surface of the region, where the marking includes at least one active agent.

In another embodiment, there is provided a confectionery composition including a region including at least one demulcent and a marking on the exterior of the region, where the marking includes at least one active agent and is substantially free of the demulcent.

In another embodiment, there is provided a method of delivering an active agent to an individual including the steps of providing a confectionery composition which includes a region and a marking on the exterior surface of the region, where the marking includes at least one active agent, and administering the confectionery composition into the oral cavity of the individual.

In other embodiments, there is provided a method of delivering two different active agents to an individual, including the steps of providing a confectionery composition, which includes a region including a first active agent and a marking on the exterior surface of the region, where the marking includes a second active agent and is substantially free of the first active agent, and administering the confectionery composition into the oral cavity of the individual.

In still other embodiments, there is provided a method of making a confectionery composition including the steps of forming a region which includes a first confectionery composition, preparing a second confectionery composition, which includes at least one active agent, applying the second confectionery composition to the region to form a marking on the exterior surface of the region, and allowing the composition to harden.

In another embodiment, there is provided a method for delivering an active agent to an individual, which includes the steps of providing a chewing gum that includes a chewing gum region and at least one marking, where the marking includes at least one active agent, and chewing the chewing gum to cause the active agent to be released from the chewing gum into the buccal cavity of the individual.

In another embodiment, there is provided a method for reducing the amount of active agent necessary to achieve an effect in an individual as compared to a typical method of introducing an active agent, which includes the steps of providing a chewing gum including a chewing gum region and a marking, where the marking includes at least one active agent that is typically present in the chewing gum region of a chewing gum composition, and the marking includes less than the typical amount of the active agent that is normally present to achieve a specific effect, chewing the chewing gum and thereby causing the active agent to be released into the saliva of the individual, and continuing to chew the chewing gum, forcing the active agent through an oral mucosa contained in a buccal cavity of the individual.

### DETAILED DESCRIPTION

Embodiments described herein provide confectionery compositions that can deliver active agents to the mouth more efficiently than conventional confectionery compositions. In some embodiments described herein, there is provided confectionery compositions including a first confectionery region and at least one marking on the exterior surface of the region, and where the marking includes at least one active agent. The distinct regions and markings contribute to the efficiency of the delivery of the active to the consumer. The marking may partially cover the surface of the confectionery region, and may be continuous or discontinuous. As used herein “marking” refers to a composition as described herein that is applied to or otherwise is present on or in the external surface of the confectionery region. “Markings” may include a composition that is embedded in the confectionery region, which is applied to the surface of the confectionery region, which is adhered to the external surface of the confectionery region, or is somehow connected with the external surface of the confectionery region. The marking may include any desired composition, and includes, but not is limited to, chewing gum, (which includes bubble gum), chocolate, chewy candy, hard candy, boiled candy, breath and other oral care film, gummy candy, jelly, fudge, caramel, taffy, gelatin, nougat, fondant, or combinations of one or more of these.

With certain active agents, such as cooling agents, the confectionery compositions of the present invention may provide as much as a 50-fold more efficient delivery of active agents to the oral cavity of a consumer and still achieve the same cooling sensation as conventional compositions. Including a smaller amount of certain active agents can be important, as various active agents may potentially irritate the user when present in large amounts. For instance, smaller amounts of potentially irritating cooling agents can be delivered to the oral cavity and throat of the consumer. Moreover, the smaller quantities of expensive active agents results in lower production costs. Non-limiting examples of active agents for use herein include flavor agents, sensates, demulcents, sweeteners, taste modifiers, coloring agents, functional agents, pharmaceutical agents or medicaments, breath freshening agents, and dental care agents. In some embodiments, the active agent is present in an amount from about 0.5 mg to about 1 g. Alternatively, the active agent may be present in an amount less than about 50 mg, less than about 10 mg, less than about 5 mg, less than about 1 mg, and less than about 0.5 mg, depending on the individual consumer's desire.

The reduced entrapment of active agents in the confectionery composition has numerous advantages, including less oral cavity irritation from active agents, such as flavors or cooling agents, and smaller quantities of active agents needed to obtain a desired level of the particular effect. Use of lesser quantities of active agents also has the advantage of lowering production costs.

Embodiments described herein are suitable for any confectionery composition known in the art. As used herein, the term “confectionery composition” includes, but is not limited to, chewing gum, (which includes bubble gum), chocolate, lozenges, mints, tablets, chewy candies, hard candies, boiled candies, breath and other oral care films or strips, candy canes, lollipops, gummies, jellies, fudge, caramel, hard and soft panned goods, toffee, taffy, gelatin candies, gum drops, jelly beans, nougats, fondants, or combinations of one or more of these, or edible compositions incorporating one or more of these. Particularly desirable embodiments for the confectionery composition include hard candy, throat lozenges, chewing gum, bubble gum, center filled candy, or soft chewy candy.

The confectionery compositions described herein may additionally contain a coating such as a clear or translucent candy overcoating. In some embodiments, the confectionery composition may contain a center-fill composition within the confectionery region. The coating and the center-fill composition may include a variety of optional components, such as sweeteners and flavors, and may further include at least one active agent. The coating and the center-fill composition may optionally be sugar-free. Likewise, the marking and the confectionery region may optionally be sugar-free.

In some embodiments, a hard candy is provided, where the marking includes at least one stripe applied to the exterior of the confectionery region. In some embodiments, the marking may be substantially free of pectin or other demulcent. In these embodiments, the confectionery region of the candy may include a demulcent. Both the marking and the confectionery region may optionally contain other ingredients commonly found in confectionery compositions, such as sugar or other sweeteners, flavors, coloring additives, and fillers. Examples of flavors, sweeteners, and other useful additives are discussed in more detail hereinbelow.

In some embodiments, active agents may be employed, containing substances with medicinal activity or drug substances. Such active agents may be added to the confectionery region, the marking, or both. Active agents may further be included in other optional layers or regions, including coatings or center-fill compositions. In some embodiments employing center-fill candies, the active agent may be located in the confectionery region part of the confectionery composition, and other components, such as demulcents, may be located in the center-fill region.

### Marking

The marking of the confectionery composition desirably contains at least one active agent. The active agent may be any active agent desired, including those described in more detail below, and includes, for example, a flavor agent. The marking may contain sweeteners or other flavoring components, and may optionally be sugared or sugar free. In some embodiments, the confectionery region may contain one or more active agents, which may be different than the first active agent contained in the marking.

The marking on the external surface of the confectionery region may be continuous or discontinuous, and may be formed as an indent into the confectionery region, or may be formed as a bump or ridge on the outer surface of the region. Non-limiting examples of the marking include one or more stripes, dots, letters, shapes, such as circles, squares, triangles, and the like, symbols, finger-like markings surrounding the perimeter of the confectionery region, swirls, fanciful designs, random designs, and combinations thereof, such as letters in circles, or stripes in triangles. In some embodiments, the marking may be a discontinuous plurality of markings. If the markings include letters or symbols, the letters or symbols may spell a word, such as a brand name, or provide a symbolic reference to connote a specific meaning or communication. If the marking includes a fanciful design or symbol, it may include dots, swirls, or other markings, either randomly disposed on the confectionery region, or as part of a pattern or both. The markings may also function to provide information about the product, such as the flavor or other information. The markings may contain several stripes or one stripe, or there may be a continuous stripe around the confectionery region, such as a spiral. In other embodiments, the markings may resemble sports equipment, such as footballs, helmets, baseballs, bats, basketballs, hoops, soccer balls, and the like. In other embodiments, the markings may resemble other real-life physical objects, such as books, movie cameras, animals, or any other object desired.

In some embodiments, the marking may be formed as an indent into the external surface of the confectionery region, such as a v-shaped indent, convex circles, and scratch-like markings. In another embodiment, the marking may be formed as a raised surface, such as a ridge, concave circle, or several bridge regions on the external surface of the confectionery region. The marking may additionally be formed as a combination of ridges and indents on the external surface of the confectionery region. Alternatively, the marking may be continuous with the external surface of the confectionery region, forming a relatively smooth texture on the surface. In such embodiments, the marking and the confectionery region are in contact but are not mixed. Optionally, the confectionery region and marking may be of different colors.

As mentioned above, the marking desirably contains at least one active agent, which will be described in more detail below. The marking may be formed as a discrete area of the confectionery product, such that the marking is not co-extant with any components, such as active agents or flavors, in the confectionery region. The physical separation of the marking and confectionery region may help to reduce the degree of entrapment of active agents that may be present in the confectionery region. For example, entrapment of components in the confectionery region may particularly pose a problem where the confectionery region contains a demulcent. Demulcents can entrap the active agents, such as flavors, employed in some embodiments, requiring the use of larger quantities of active agents than would otherwise be necessary if there was no entrapment effect. This separation permits the use of smaller quantities of active agents to achieve a particular effect. On contact with the saliva, the active agent in the marking is rapidly released, allowing for faster delivery of the sensory signal to the trigeminal area of the oral cavity, with the use of smaller quantities of active agents than in conventional products with flavors or other active agents uniformly distributed therein. In some embodiments, for instance, it may be more preferential to use smaller quantities of active agents, which may produce an unpleasant taste if present in high amounts.

The marking may be applied to the exterior of the confectionery composition by any means desired. For example, the marking may be applied to the exterior of the confectionery composition via extrusion means or via a printing process. Other methods for applying the marking to the external surface of the confectionery region are contemplated. The marking may be in the form of a solid, liquid or semi-solid composition, and may be either water or oil based as it is applied to the external surface of the confectionery region. The marking may include a low melting fat or wax for application. Fats may include both fats and oils, either saturated or unsaturated, and includes lipids. Suitable waxes include edible waxes, such as bees wax, carnauba wax, candelilla wax and the like. Alternatively, the marking may be embedded within the body of the confectionery composition, but not mixed with the confectionery region. Alternatively, the marking may be adhered to the external surface of the confectionery region. In some embodiments, the marking may be substantially free of demulcents.

In some embodiments, there may be a plurality of markings on the external surface of the confectionery region. The markings may be independently continuous or discontinuous, and may overlap.

In some embodiments, the marking may cover less than 100% of the external surface of the confectionery region. In some embodiments, the marking covers about 0.01% to less than about 75% of the surface of the confectionery region. The marking may cover less than about 25% of the surface area of the confectionery region, or it may optionally cover less than about 10% of the surface area of the confectionery region, less than about 5% of the surface area of the confectionery region, less than about 1% of the surface area of the confectionery region, less than about 0.5% of the surface area of the confectionery region, less than about 0.1% of the surface area of the confectionery region, or more specifically, less than about 0.05% of the surface area of the confectionery region.

In some embodiments, the marking may be about 0.01% to less than about 50% by weight of the confectionery composition. In alternative embodiments, the marking may be present in amounts of less than about 10% by weight of the composition, or it may be present in amounts of less than about 5% by weight of the confectionery composition, less than about 1% by weight of the confectionery composition, less than about 0.5% by weight of the confectionery composition, or, more specifically, in amounts less than about 0.1% by weight of the confectionery composition.

In some embodiments, the marking may contain various amounts of active agents. For example, a first active agent contained in the marking may be present in a ratio of about 10 to 1 by weight as compared to a second active agent in the confectionery region, and vice versa. The active agent in the marking may be different or the same as other active agents that may be present in other areas of the confectionery composition, including any active agents in the coating, the center-fill composition, or the confectionery region. A description of suitable active agents for use in the marking and optionally in other regions of the confectionery composition is set forth below.

### Flavor Agents

The various areas of the confectionery composition, including the confectionery region and the marking, may contain flavor agents (flavorants, flavorings, flavors). Suitable flavor agents may include natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yoghurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, e.g., melonal (melon), 2,6-dimethyloctanal (green fruit), and 2-dodecenal (citrus, mandarin), cherry, grape, blueberry, blackberry, strawberry shortcake, and mixtures thereof.

### Sensates

Particularly desirable active agents for use in some embodiments include sensate components, which may include cooling agents, warming agents, tingling agents, effervescent agents, and combinations thereof. Any area of the confectionery composition, including the confectionery region and the marking, may contain sensate components.

A variety of well known cooling agents may be employed. For example, among the useful cooling agents are included xylitol, erythritol, dextrose, sorbitol, menthane, menthone, ketals, menthone ketals, menthone glycerol ketals, substituted p menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of para-menthane, 2-mercaptocyclodecanone, hydroxycarboxylic acids with 2 to 6 carbon atoms, cyclohexanamides, menthyl acetate, menthyl salicylate, N-2,3 trimethyl-2-isopropyl butanamide (known as WS-23), N-ethyl-p-menthane-3-carboxamide (known as WS-3), isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-1-menthoxypropane-1,2-diol (from Takasago, FEMA 3784); and menthyl lactate; (from Haarman & Reimer, FEMA 3748, tradename FRESCOLAT® type ML), WS-30, WS-14, eucalyptus extract (p-mentha-3,8-diol), menthol (its natural or synthetic derivatives), menthol PG carbonate, menthol EG carbonate, menthol glyceryl ether, N-tertbutyl-p-menthane-3-carboxamide, para-menthane-3-carboxylic acid glycerol ester, methyl-2-isopropyl-bicyclo (2.2.1), Heptane-2-carboxamide; and menthol methyl ether, and menthyl pyrrolidone carboxylate among others. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol-n-butylether (TK 1000) supplied by Takasago Perfumary Company Limited, Tokyo, Japan, vanillyl alcohol n-propylether, vanillyl alcohol isopropylether, vanillyl alcohol isobutylether, vanillyl alcohol isoamylether, vanillyl alcohol n-hexylether, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, isoamylalcohol, benzyl alcohol, glycerine, and combinations thereof.

In some embodiments, a tingling sensation may be provided. One such tingling sensation is provided by adding jambu, oleoresin, or spilanthol to some examples. In some embodiments, alkylamides extracted from materials such as jambu or sanshool may be included.

Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Sensate components may also be referred to as “trigeminal stimulants” such as those disclosed in U.S. Pre-grant Publication No. 2005/0202118, which is incorporated herein by reference. Trigeminal stimulants are defined as an orally consumed product or agent that stimulates the trigeminal nerve. Examples of cooling agents which are trigeminal stimulants include menthol, WS-3, N-substituted p-menthane carboxamide, acyclic carboxamides including WS-23, WS-5, WS-14, methyl succinate, and menthone glycerol ketals. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin and menthol glycerin ether, unsaturated amides, and combinations thereof. Other cooling compounds can include derivatives of 2,3-dimethyl-2-isopropylbutyric acid such as those disclosed in U.S. Pat. No. 7,030,273, which is incorporated herein by reference.

In addition to trigeminal nerve stimulants and cooling compounds, a cooling sensation can be provided by materials exhibiting a negative heat of solution including, but not limited to, polyols such as xylitol, erythritol, dextrose, and sorbitol, and combinations thereof.

In some embodiments, sensate components are used at levels that provide a perceptible sensory experience i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

### Taste Modifiers

Taste modifiers described herein may include sweeteners, sour agents, bitter agents, astringent agents, salty taste agents, and combinations thereof. Sweeteners can include sugars, sugarless bulk sweeteners, or the like, high intensity sweeteners, or mixtures thereof. Bulk sweeteners generally are present in amounts of about 5% to about 99% by weight of the confectionery or chewing gum base composition. Suitable sugar sweeteners generally include mono-saccharides, di-saccharides and poly-saccharides such as but not limited to, sucrose (sugar), dextrose, maltose, dextrin, xylose, ribose, glucose, mannose, galactose, fructose (levulose), invert sugar, sucromalt, corn syrups, maltodextrins, oligo saccharide syrups, fructo oligo saccharide syrups, partially hydrolyzed starch, corn syrup solids, resistant starches, and mixtures thereof.

Suitable sugarless bulk sweeteners include sugar alcohols (or polyols) such as, but not limited to, sorbitol, xylitol, mannitol, galactitol, maltitol, hydrogenated isomaltulose (ISOMALT), lactitol, erythritol, hydrogenated starch hydrolysates, and mixtures thereof.

Suitable hydrogenated starch hydrolysates include those disclosed in U.S. Pat. No. 4,279,931 and various hydrogenated glucose syrups and/or powders which contain sorbitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

In some embodiments, high-intensity sweeteners also may be included as sweetening agents in the compositions. Without being limited to particular sweeteners, representative categories and examples include:

(a) water-soluble sweetening agents such as dihydrochalcones, monellin, steviosides, lo han quo, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame), N—[N-(3,3-dimethylbutyl)-L-α-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose or Splenda™; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro-4,6,1′,6′-tetradeoxygalacto-sucrose; and 4,6,1′,6′-tetradeoxy-sucrose, and mixtures thereof;

(e) protein based sweeteners such as thaumaoccous danielli (Thaumatin I and II) and talin;

(f) the sweetener monatin (2-hydroxy-2-(indol-3-ylmethyl)-4-aminoglutaric acid) and its derivatives; and

(g) the sweetener Lo han guo (sometimes also referred to as “Lo han kuo”).

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycitol syrups, polyglycitol powders, lactitol, and combinations thereof.

The taste modifier (e.g., a sweetener) may be used in amounts necessary to impart the desired effect associated with use of the active component (e.g., sweetness). In general, an effective amount of intense taste modifier may be utilized to provide the level of taste modification desired, which may vary with the sweetener selected. The intense taste modifier may be present in amounts from about 0.001% to about 3%, by weight of the composition, depending upon the taste modifier or combination of taste modifiers used. The exact range of amounts for each type of taste modifiers may be selected by those skilled in the art. Various areas of the confectionery composition, including the confectionery region, the marking, and optionally, the coating and the center-fill composition may include different taste modifiers, or they may include the same taste modifiers.

### Coloring Agents

In some embodiments, one or more colorants (color agents, colors) can be included in the confectionery region, the marking, or both. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These consist of the aluminum salts of FD&C dyes extended on an insoluble substrate of alumina hydrate. Additionally, in some embodiments, certified colors can be included as calcium salts.

### Demulcents

Demulcents useful as active agents may include pectin or polymeric materials that moisturize or soothe irritated mouth or throat tissues. The demulcents may also have humectant activity, meaning that the substance absorbs moisture and moisturizes tissues in contact with the humectant substance. Any area of the confectionery composition described herein may include demulcents, including the confectionery region and the markings. In some embodiments, demulcents are not present in the marking, but may be if desired.

Demulcents useful herein may include hydrocolloid materials that hydrate and adhere to oral surfaces to provide a sensation of mouth or throat moistening. Hydrocolloid materials can include naturally occurring materials such as plant exudates, seed gums, and seaweed extracts or they can be chemically modified materials such as cellulose, starch, or natural gum derivatives. In some embodiments, hydrocolloid materials can include pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, bacterial gums, and combinations thereof. Additionally, in some embodiments, modified natural gums such as propylene glycol alginate, carboxymethyl locust bean gum, low methoxyl pectin, and their combinations can be included. In some embodiments, modified celluloses can be included such as microcrystalline cellulose, carboxymethylcellulose (CMC), methylcellulose (MC), hydroxypropylmethylcellulose (HPMC), and hydroxypropylcellulose (HPC), and combinations thereof.

Similarly, demulcents which can provide a perception of mouth hydration may be included. Such demulcents can include, but are not limited to glycerol, sorbitol, polyethylene glycol, erythritol, and xylitol.

Additionally, in some embodiments, the demulcents may be fats or lipids. Such fats can include medium chain triglycerides, vegetable oils, fish oils, mineral oils, and combinations thereof. Desirably, fats used herein may be high melting, hydrogenated, or gelled fats. In some embodiments, normally low melting fats are hydrogenated so the fats are solid at room temperature. Alternatively, the demulcents may be monoglycerides or polyglycerol fatty acid esters. Such monoglycerides and polyglycerol fatty acid esters help promote the setting ability of liquid oils, as well as forming gels. This effect can be very significant for gel strength (up to a 500-fold increase) with minimal increase in melting point of the gelled oil.

Demulcents may be treated so that they are present as a solid at room temperature. Alternatively, the demulcent may be in a liquid form at room temperature.

### Functional Agents

In some embodiments, the confectionery compositions may include one or more functional agents, including surfactants, breath freshening agents, pharmaceutical agents, nutrition supplements, oral care agents, throat care agents, and combinations thereof. The functional agents may be added to any area of the confectionery composition, including particularly in the marking, the confectionery region, or both. Pharmaceutical agents contemplated for use herein may include, but are not limited to, throat soothing agents, analgesics, anesthetics, antiseptics, cough suppressants, antitussives, expectorants, antihistamines, mucolytics, and nasal decongestants. In addition, other pharmaceutical agents, as discussed below, may be employed herein.

Throat soothing ingredients may include analgesics, anesthetics, antiseptic, and combinations thereof. In some embodiments, analgesics or anesthetics may include menthol, phenol, hexylresorcinol, benzocaine, dyclonine hydrochloride, benzyl alcohol, salicyl alcohol, and combinations thereof. In some embodiments, antiseptic ingredients may include cetylpyridinium chloride, domiphen bromide, dequalinium chloride, and combinations thereof.

Throat soothing agents include honey, propolis, aloe vera, green or red pepper extract, glycerine, menthol and combinations thereof.

Cough suppressants may fall into two groups: those that alter the consistency or production of phlegm such as mucolytics and expectorants; and those that suppress the coughing reflex such as codeine (narcotic cough suppressants), antihistamines, dextromethorphan and isoproterenol (non-narcotic cough suppressants). In some embodiments, ingredients from either or both groups may be included.

In still other embodiments, antitussives may be used, and include, but are not limited to, the group consisting of codeine, dextromethorphan, dextrorphan, diphenhydramine, hydrocodone, noscapine, oxycodone, pentoxyverine and combinations thereof.

In some embodiments, antihistamines may be added, and include, but are not limited to, acrivastine, azatadine, brompheniramine, chlorpheniramine, clemastine, cyproheptadine, dexbrompheniramine, dimenhydrinate, diphenhydramine, doxylamine, hydroxyzine, meclizine, phenindamine, phenyltoloxamine, promethazine, pyrilamine, tripelennamine, triprolidine and combinations thereof. In some embodiments, non-sedating antihistamines may include, but are not limited to, astemizole, cetirizine, ebastine, fexofenadine, loratidine, terfenadine, and combinations thereof.

In some embodiments, expectorants may be added, and include, but are not limited to, ammonium chloride, guaifenesin, ipecac fluid extract, potassium iodide and combinations thereof. In some embodiments, mucolytics may be added, and include, but are not limited to, acetylcycsteine, ambroxol, bromhexine and combinations thereof. In some embodiments, analgesic, antipyretic and anti-inflammatory agents may be added, and include, but are not limited to, acetaminophen, aspirin, diclofenac, diflunisal, etodolac, fenoprofen, flurbiprofen, ibuprofen, ketoprofen, ketorolac, nabumetone, naproxen, piroxicam, caffeine and mixtures thereof. In some embodiments, local anesthetics may include, but are not limited to, lidocaine, benzocaine, phenol, dyclonine, benzonotate and mixtures thereof.

In some embodiments nasal decongestants and ingredients that provide the perception of nasal clearing may be included. In some embodiments, nasal decongestants may include but are not limited to phenylpropanolamine, pseudoephedrine, ephedrine, phenylephrine, oxymetazoline, and combinations thereof.

A variety of nutritional supplements may also be used as active ingredients including virtually any vitamin or mineral. For example, vitamin A, vitamin C, vitamin D, vitamin E, vitamin K, vitamin B6, vitamin B12, thiamine, riboflavin, biotin, folic acid, niacin, pantothenic acid, sodium, potassium, calcium, magnesium, phosphorus, sulfur, chlorine, iron, copper, iodine, zinc, selenium, manganese, choline, chromium, molybdenum, fluorine, cobalt and combinations thereof, may be used.

Examples of nutritional supplements that may be used as active ingredients are set forth in U.S. Publication Nos. 2003/0157213 A1, 2003/0206993 and 2003/0099741 A1 which are incorporated in their entirety herein by reference for all purposes.

Various herbals may also be used as active ingredients such as those with various medicinal or dietary supplement properties. Herbals are generally aromatic plants or plant parts and or extracts thereof that can be used medicinally or for flavoring. Suitable herbals may be used singly or in various mixtures. Commonly used herbs include Echinacea, Goldenseal, Calendula, Rosemary, Thyme, Kava Kava, Aloe, Blood Root, Grapefruit Seed Extract, Black Cohosh, Ginseng, Guarana, Cranberry, Gingko Biloba, St. John's Wort, Evening Primrose Oil, Yohimbe Bark, Green Tea, Ma Huang, Maca, Bilberry, Lutein, and combinations thereof.

### Breath Freshening Agents

Some embodiments set fort herein may include breath fresheners, which may include essential oils as well as various aldehydes, alcohols, and similar materials. In some embodiments, essential oils may include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde may be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments breath fresheners may include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof.

In some embodiments, the release profiles of probiotics can be managed for a confectionery composition including, but not limited to lactic acid producing microorganisms such as Bacillus coagulans, Bacillus subtilis, Bacillus laterosporus, Bacillus laevolacticus, Sporolactobacillus inulinus, Lactobacillus acidophilus, Lactobacillus curvatus, Lactobacillus plantarum, Lactobacillus jenseni, Lactobacillus casei, Lactobacillus fermentum, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn,™ Actizol,™ and Nutrazin.™ Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

Breath freshening agents may be incorporated into any area of the confectionery composition, including, for example, in the marking, the confectionery region, center-fill, coating, or combinations thereof.

### Dental Care Agents

Some embodiments described herein may include dental care components (also known as oral care ingredients), which may be included in any area of the confectionery composition, such as the marking and the confectionery region. Such dental care components may include but are not limited to tooth whiteners, stain removers, oral cleaning, bleaching agents, desensitizing agents, dental remineralization agents, antibacterial agents, anticaries agents, plaque acid buffering agents, surfactants and anticalculus agents. Non-limiting examples of such ingredients may include, hydrolytic agents including proteolytic enzymes, abrasives such as hydrated silica, calcium carbonate, sodium bicarbonate and alumina, other active stain-removing components such as surface-active agents, including, but not limited to anionic surfactants such as sodium stearate, sodium palminate, sulfated butyl oleate, sodium oleate, salts of fumaric acid, glycerol, hydroxylated lecithin, sodium lauryl sulfate and chelators such as polyphosphates, which are typically employed as tartar control ingredients. In some embodiments, dental care ingredients may also include tetrasodium pyrophosphate and sodium tri-polyphosphate, sodium bicarbonate, sodium acid pyrophosphate, sodium tripolyphosphate, xylitol, sodium hexametaphosphate.

In some embodiments, peroxides such as carbamide peroxide, calcium peroxide, magnesium peroxide, sodium peroxide, hydrogen peroxide, and peroxydiphospate are included. In some embodiments, potassium nitrate and potassium citrate are included. Other examples may include casein glycomacropeptide, calcium casein peptone-calcium phosphate, casein phosphopeptides, casein phosphopeptide-amorphous calcium phosphate (CPP-ACP), and amorphous calcium phosphate. Still other examples may include papaine, krillase, pepsin, trypsin, lysozyme, dextranase, mutanase, glycoamylase, amylase, glucose oxidase, and combinations thereof.

Further examples may include surfactants such as sodium stearate, sodium ricinoleate, and sodium lauryl sulfate surfactants for use in some embodiments to achieve increased prophylactic action and to render the dental care ingredients more cosmetically acceptable. Surfactants can preferably be detersive materials which impart to the composition detersive and foaming properties. Suitable examples of surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

In addition to surfactants, dental care ingredients may include antibacterial agents such as, but not limited to, triclosan, chlorhexidine, zinc citrate, silver nitrate, copper, limonene, and cetyl pyridinium chloride. In some embodiments, additional anticaries agents may include fluoride ions or fluorine-providing components such as inorganic fluoride salts. In some embodiments, soluble alkali metal salts, for example, sodium fluoride, potassium fluoride, sodium fluorosilicate, ammonium fluorosilicate, sodium monofluorophosphate, as well as tin fluorides, such as stannous fluoride and stannous chloride can be included. In some embodiments, a fluorine-containing compound having a beneficial effect on the care and hygiene of the oral cavity, e.g., diminution of enamel solubility in acid and protection of the teeth against decay may also be included as an ingredient. Examples thereof include sodium fluoride, stannous fluoride, potassium fluoride, potassium stannous fluoride (SnF.sub.2-KF), sodium hexafluorostannate, stannous chlorofluoride, sodium fluorozirconate, and sodium monofluorophosphate. In some embodiments, urea may be included.

### Methods of Preparation

Some embodiments provided herein relate to methods of preparing the confectionery compositions. In accordance therewith, in some embodiments there is first provided a confectionery region. The confectionery region may be prepared by conventional techniques for preparing confectionery compositions, including via extrusion, boiling, or other known methods for forming the confectionery region. The region may be a center-filled confectionery region, and may alternatively be hard or soft candy, or the confectionery region may be a gum, as described above. The confectionery region may be of any shape or size desired, and may be shaped as, for example, a pellet, tablet, ball, pillow, chunk, stick and slab, among others. Additional methods will be known to persons of skill in the art of making confectionery compositions.

A second confectionery composition, which contains at least one active agent, may be separately prepared by any known method. The second confectionery composition may be the same as the confectionery region, or it may be different. For example, the confectionery region may be a chewing gum, while the second confectionery composition is a candy. In some embodiments, a second confectionery composition may include dispersion stabilizers, film formers, binders, surfactants, thickening agents and/or adhesion enhancers as commonly known in the art. The second confectionery composition will form a marking on the confectionery region. The second confectionery composition (marking) is applied to the external surface of the confectionery region. The marking may be applied by techniques known in the art of confectionery making. For example, the marking may be applied as a liquid through a fine nozzle at the surface of the confectionery region or via a drizzle process. Alternatively, the marking may be applied via extrusion of the second confectionery composition onto the external surface of the confectionery region. The marking may be either pigmented or non-pigmented and may be aqueous or fat- or oil based. In some embodiments, the marking may be applied via etching or other indent-creating methods, or it may be applied via adhesion methods to create a raised texture. Multiple layers of the marking may be applied to the confectionery region. The marked confectionery composition is then allowed to harden.

Alternatively, the marking may be applied to the surface of a confectionery region via a printing process. In such applications, an inkjet printing apparatus may be used, including, for example, continuous jet and drop-on-demand printing systems. A piezoelectric inkjet printing system may be used if desired. Other printing systems include piezojet and bubblejet printheads. Alternatively, other printing apparatuses such as offset gravure printing or other roller printing, silk screen and pad printing, spray printing, and porous transfer sheets may be used. Generally, the marking applied by a printing process is wax-based or fat-based, but may include water-based markings including adhesives and other such markings.

Optionally, during the forming steps, grooves or indents may be etched onto the outer surface of the confectionery region. The second confectionery composition may then be applied to the surface by means that will allow the second confectionery composition to be present in the grooves or indents.

Additionally, the entire confectionery region may be optionally coated with a coating. The coating layer may contain any ingredients conventionally used in the confectionery art. For instance, the coating may contain sugar, polyols or high intensity sweeteners or the like, coloring agents, flavor agents and warming and/or cooling agents, among others. The coating may optionally include an active agent, which may be the same or different than other active agents incorporated into the other areas of the confectionery composition.

### Methods of Delivery of Actives

Embodiments described herein also provide methods of delivering at least one active agent to an individual. In some embodiments, there is provided a confectionery composition as described above. The confectionery composition includes a confectionery region and at least one marking on the exterior surface of the confectionery region. Desirably the marking contains at least one active agent, and optionally may contain more than one active agent if desired. The confectionery composition is introduced into the oral cavity of the individual, where the active agent is released, either through contact with saliva in the oral cavity, or via mastication of the individual, releasing the active agent.

In an alternative method of delivery, there is provided a method of delivering at least two different active agents to an individual. In this method, there is provided a confectionery composition, prepared as described above. The confectionery composition includes a confectionery region, which includes a first active agent. Applied to the exterior surface of the confectionery region is at least one marking, where the marking contains at least one active agent. Desirably, the marking contains a different active agent than that present in the confectionery region, and is devoid of the first active agent present in the confectionery region. Alternatively, there may be more than one active agent in the confectionery region or in the marking. Additionally, there may be multiple markings on the surface of the confectionery region, each marking containing an active agent, which may be the same or different than the active agent contained in the first marking. The confectionery composition is introduced into the oral cavity of the individual, where the active agents are released, either through contact with saliva in the oral cavity, or via mastication of the individual, releasing the active agents.

In some embodiments, the active agent(s) may be delivered to an individual by providing a chewing gum composition including a chewing gum region and marking, where the marking contains at least one active agent as described above. The individual may then chew the chewing gum composition, which causes the active agent to be released from the chewing gum and into the buccal cavity of the individual. In some embodiments, the active agent may be present in an amount less than the amount typically included in chewing gum compositions. In such embodiments, the marking contains at least one active agent in an amount less than that typically required to achieve the same desired effect in the individual. The individual then may chew the chewing gum composition, releasing the active agent into the saliva of the individual, and forcing the active agent through the oral mucosa contained in a buccal cavity of the individual. The use of lesser amounts of active agents may be useful in maintaining low production costs. Further, when active agents are present in high amounts, they may irritate the individual. Use of lesser amounts of active agents, therefore, also may help curb this irritation.

In any of the above methods of delivering active agents, there may be various amounts of active agent present in the confectionery composition, depending on the active used and the effect desired. In some embodiments, the confectionery composition contains a total of less than about 10 mg of active agent, and may contain less than about 5 mg, less than about 1 mg, and more specifically, less than about 0.5 mg of active agent. Desirably the active agent is present in an amount that is less than about 10% of the amount that is typically required in a confectionery composition to achieve an equivalent effect.

### EXAMPLES

### Example 1

### Striped Hard-Boiled Candy

Table 1 provides a formula for a confectionery composition with demulcent and cooling agents. The comparative confectionery composition (designated as Example 1A) is a confectionery composition incorporating cooling agent uniform throughout in the confectionery region and no cooling agent in a stripe marking. In this comparative confectionery composition, the composition requires around 0.2-1.5 g (e.g., 0.818 g) of cooling agent to achieve the desired sensory effect. The inventive composition (designated as Example 1B) incorporates cooling agent in a stripe marking, and no cooling agent in the confectionery region. The amount of cooling agent required in the inventive composition to achieve the same sensory perception is only around 0.001-0.1 g (e.g., 0.0325 g). A substantially smaller amount of cooling agent in the composition results in lower production costs, while also having the advantage of smaller quantities of potentially irritating cooling agents introduced into the oral cavity of the user.

TABLE 1

| | 1A. Control | | 1B. Cooling Agent | |
|---|---|---|---|---|
| Component | Confection (g) | | In Stripe (g) | |
| CONFECTIONERY | | | | |
| REGION | | | | |
| Sugar Mixture | 540-675 | (e.g., 595) | 540-675 | (e.g., 595) |
| Fat(s) | 0-15 | (e.g., 9.62) | 0-15 | (e.g., 9.62) |
| Demulcent(s) | 0.5-5 | (e.g., 1.26) | 0.5-5 | (e.g., 1.26) |
| Color solution(s) | 0.5-5 | (e.g., 1.4) | 0.5-5 | (e.g., 1.4) |
| Acid(s) | 0.5-5 | (e.g., 3.5) | 0.5-5 | (e.g., 3.5) |
| Flavor(s) | 0.2-1.5 | (e.g., 0.82) | 0.2-1.5 | (e.g., 0.82) |
| Cooling Agent(s) | 0.2-1.5 | (e.g., 0.818) | 0 | |
| | Take 20 g of candy | | Take 20 g of candy | |
| | base and add white | | base and add white | |
| WHITE STRIPE | color | | color | |
| Color solution | 0-1 | (e.g., 0.512) | 0-1 | (e.g., 0.512) |

| | | | |
|---|---|---|---|
| Cooling Agents | 0 | 0.001-1 | (e.g., 0.0325) |

To form the confectionery compositions according to the formulae set forth above, the sugar and water are first cooked at about 140-150° C., and the demulcent solution is added. After adding the demulcent, the fat, acids, cooling compounds, flavor and color are added. For the stripe material, 20 g of the liquid candy mass is removed and mixed with the color solution. For example 1B, the candy mass portion for the striping was removed before the demulcent solution was added, and cooling compounds were added only to the striping portion, not to the main confectionery region.

The confectionery region is then spread on a tempering band to reduce its temperature and enhance the phase shift to an amorphous phase. The cooled candy is recovered at the end of the tempering table in the form of a rope.

The rope is then pulled vertically through a batch former and extruded concurrently with the white stripe material. The white stripe material is delivered through a nozzle and applied to the exterior surface of the candy rope.

The striped rope is then passed through a die cutting apparatus to form individual pieces of confectionery composition at the desired piece weight. The striped confectionery pieces are then passed through a cooling tunnel to fully solidify the candy.

### Example 2

### Striped Chewing Gum

### Slab

Chewing gum compositions are prepared in accordance with Table 2 below. Comparative example 2A describes the composition of an unstriped chewing gum composition, whereas inventive example 2B sets forth the composition of a striped chewing gum composition of the present invention.

TABLE 2

| | Comparative | Inventive |
|---|---|---|
| | Example 2A | Example 2B |
| | Unstriped | Striped |
| Component | (% in formula) | (% in formula) |
| Gum Composition | | |
| Gum base | 15-55 | 15-55 |
| Acetylated monoglycerides | 0.1-1 | 0.1-1 |
| Lecithin | 0.1-1 | 0.1-1 |
| Sweetener(s) | 30-80 | 30-80 |
| Glycerin | 2-15 | 2-15 |
| Flavor(s) | 0.5-6 | 0.5-6 |
| Cooling compound(s) | 0.2-2 | — |
| Intense Sweeter(s) | 2-10 | 2-10 |
| (both encapsulated and un- | | |
| encapsulated) | | |
| STRIPE Composition | | |
| Stripes | — | 1-10 |
| Cooling compounds | | 0.001-1 |

### Method of Preparation of Unstriped Chewing Gum

### Comparative Example 2A

Chewing gum base is first melted at a temperature of about 150-175° F. The molten gum base is placed in a standard mixer and the remaining ingredients (sweeteners, lecithin, intense sweeteners and flavor/cooling blends) are added and thoroughly mixed for about 1 to 20 minutes. The resulting mix is formed into long slabs via rolling, extrusion or other forming techniques followed by cutting into sticks, casting into pellets and then may be coated, or pressed into tablets, among other chewing gum forms.

### Method of Preparation of Striped Chewing Gum

### Comparative Example 2B

A chewing gum with cooling agent in the stripes in accordance with the present invention is made from a gum composition batch and a stripe composition batch, prepared in a 20:1 ratio by weight. The gum composition batch is prepared in the same manner as described in comparative example 2A with the same ingredients, except for the cooling compounds. A cooling stripe composition is prepared in the same manner as the comparative composition, incorporating a cooling agent in the composition.

A slab of gum composition batch is rolled to a desired thickness and an indentation is introduced to the surface of the slab. A thin rope of stripe composition batch is fitted into the indentation and pressed firmly so that the stripe composition is flush with the surface of the gum composition slab. The striped composition is then cut to a desired piece size.

1. A confectionery composition comprising:
(a) a confectionery region; and
(b) a marking on the exterior surface of said region, wherein said marking comprises at least one active agent.

2. The confectionery composition of claim 1, wherein said marking is discontinuous.

3. The confectionery composition of claim 1, wherein said marking is selected from the group consisting of stripes, dots, letters, fanciful designs, and combinations thereof.

4. The confectionery composition of claim 1, wherein said marking is a discontinuous plurality of markings.

5. The confectionery composition of claim 1, wherein said at least one active agent comprises more than one different active agents.

6. The confectionery composition of claim 1, wherein said marking comprises at least one stripe.

7. The confectionery composition of claim 1, wherein said marking is indented within said confectionery region.

8. The confectionery composition of claim 7, wherein the exterior surface of said confectionery region is continuous with said marking.

9. The confectionery composition of claim 1, further comprising a coating.

10. The confectionery composition of claim 1, wherein said confectionery region comprises a center-filled composition.

11. The composition of claim 1, wherein said active agent is selected from the group consisting of sensates, demulcents, flavors, taste modifiers, functional agents, and combinations thereof.

12. The composition of claim 11, wherein said sensate is selected from the group consisting of cooling agents, warming agents, tingling agents, effervescent agents, and combinations thereof.

13. The composition of claim 11, wherein said demulcent is selected from the group consisting of pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, bacterial gums, propylene glycol alginate, carboxymethyl locust bean gum, low methoxyl pectin, microcrystalline cellulose, carboxymethylcellulose, methylcellulose, hydroxypropylmethylcellulose, hydroxypropylcellulose, glycerol, sorbitol, polyethylene glycol, erythritol, xylitol, medium chain triglycerides, vegetable oils, fish oils, mineral oils, and combinations thereof.

14. The composition of claim 11, wherein said demulcent is treated so that it is a solid at room temperature.

15. The composition of claim 11, wherein said demulcent is selected from a monoglyceride or polygylcerol fatty acid ester.

16. The composition of claim 11, wherein said taste modifier is selected from the group consisting of sweeteners, sour agents, bitter agents, astringent agents, salty taste agents, and combinations thereof.

17. The composition of claim 11, wherein said functional agent is selected from the group consisting of surfactants, breath freshening agents, pharmaceutical actives, nutrition supplements, oral care agents, throat care agents, and combinations thereof.

18. The composition of claim 1, further comprising a second active agent in a part selected from the group consisting of said confectionery region, a coating, a center-filled composition within said confectionery region and combinations thereof.

19. The composition of claim 18, wherein said second active agent is different than said active agent in said marking.

20. The composition of claim 18, wherein the ratio of the amount by weight of said active agent in said marking to the amount by weight of said second active agent is about 10:1 to 1:10.

21. The composition of claim 18, wherein said second active agent is the same as said active agent in said marking.

22. The composition of claim 1, further comprising two or more active agents in a part of the composition selected from the group consisting of said confectionery region, a coating, and a center-filled composition within said confectionery region, wherein the active agent present in any part containing an active agent is different from the active agent present in any other part containing an active agent.

23. The composition of claim 1, wherein said confectionery region is sugar-free.

24. The composition of claim 1, wherein said marking is sugar-free.

25. The composition of claim 9, wherein said coating is sugar-free.

26. The composition of claim 10, wherein said center filled composition is sugar-free.

27. The composition of claim 1, wherein said marking covers about 0.01% to less than about 75% of the surface area of said confectionery region.

28. The composition of claim 1, wherein said marking covers less than about 25% of the surface area of said confectionery region.

29. The composition of claim 1, wherein said marking covers less than about 10% of the surface area of said confectionery region.

30. The composition of claim 1, wherein said marking covers less than about 5% of the surface area of said confectionery region.

31. The composition of claim 1, wherein said marking covers less than about 1% of the surface area of said confectionery region.

32. The composition of claim 1, wherein said marking covers less than about 0.5% of the surface area of said confectionery region.

33. The composition of claim 1, wherein said marking covers less than about 0.1% of the surface area of said confectionery region.

34. The composition of claim 1, wherein said marking covers less than about 0.05% of the surface area of said confectionery region.

35. The composition of claim 1, wherein said marking comprises about 0.01% to less than about 50% by weight of the confectionery composition.

36. The composition of claim 1, wherein said marking comprises less than about 10% by weight of the confectionery composition.

37. The composition of claim 1, wherein said marking comprises less than about 5% by weight of the confectionery composition.

38. The composition of claim 1, wherein said marking comprises less than about 1% by weight of the confectionery composition.

39. The composition of claim 1, wherein said marking comprises less than about 0.5% by weight of the confectionery composition.

40. The composition of claim 1, wherein said marking comprises less than about 0.1% by weight of the confectionery composition.

41. The composition of claim 1, wherein the amount of said active agent is about 0.5 mg to about 1 g.

42. The composition of claim 1, wherein said active agent is present in amounts of less than about 50 mg.

43. The composition of claim 1, wherein said active agent is present in amounts of less than about 10 mg.

44. The composition of claim 1, wherein said active agent is present in amounts of less than about 5 mg.

45. The composition of claim 1, wherein said active agent is present in amounts of less than about 1 mg.

46. The composition of claim 1, wherein said active agent is present in amounts of less than about 0.5 mg.

47. A confectionery composition comprising:
(a) a confectionery region comprising at least one demulcent; and
(b) a marking on the exterior surface of said region, wherein said marking comprises at least one active agent and is substantially free of demulcent.

48. The composition of claim 47, wherein said marking is discontinuous.

49. The composition of claim 47, wherein said active agent is selected from the group consisting of sensates, flavors, taste modifiers, functional agents, and combinations thereof.

50. The composition of claim 47, wherein said active agent comprises a flavor.

51. The composition of claim 47, wherein said active agent comprises a cooling agent.

52. The composition of claim 47, further comprising a coating.

53. A method of delivering at least one active agent to an individual comprising the steps of:
(a) providing a confectionery composition comprising:
i. a confectionery region; and
ii. a marking on the exterior surface of said region, wherein said marking comprises at least one active agent; and
(b) administering the confectionery composition into the oral cavity of the individual.

54. The method of claim 53, wherein said at least one active agent is present in amounts of less than about 10 mg.

55. The method of claim 53, wherein said at least one active agent is present in amounts of less than about 5 mg.

56. The method of claim 53, wherein said at least one active agent is present in amounts of less than about 1 mg.

57. The method of claim 53, wherein said at least one active agent is present in amounts of less than about 0.5 mg.

58. A method of delivering two different active agents to an individual comprising the steps of:
(a) providing a confectionery composition comprising:
i. a confectionery region comprising a first active agent; and
ii. a marking on the exterior surface of said region, wherein said marking comprises a second active agent and is substantially free of said first active agent; and
(b) administering said confectionery composition into the oral cavity of the individual.

59. The method of claim 58, wherein said first active agent comprises a demulcent and said second active agent is selected from the group consisting of sensates, flavors, taste modifiers, functional agents and combinations thereof.

60. A method of making a confectionery composition comprising the steps of:
(a) forming a confectionery region comprising a first confectionery composition;
(b) preparing a second confectionery composition comprising at least one active agent;
(c) applying said second confectionery composition to said confectionery region to form a marking on the exterior surface of said region; and
(d) allowing the second confectionery composition to harden.

61. The method of claim 60, wherein said second confectionery composition is applied to said confectionery region in the form of a liquid.

62. The method of claim 61, wherein said liquid confectionery composition is applied by an extruding process.

63. The method of claim 61, wherein said liquid confectionery composition is applied by a printing process.

64. A method for delivering an active agent to an individual comprising the steps of:
(a) providing a chewing gum product comprising a chewing gum region and at least one marking, wherein said marking comprises at least one active agent; and
(b) chewing the chewing gum product thereby releasing the active agent from the chewing gum product into the buccal cavity of the individual.

65. The method of claim 64, further comprising the step of continuing to chew said chewing gum product, thereby creating a fluid pressure causing the active agent to enter the systemic system of the individual through an oral mucosa of the individual.

66. The method of claim 64, wherein said step (b) comprises chewing said chewing gum product for at least 2 minutes.
